(19) United States
(12) Patent Application Publication
(10) Pub. No.: US 2003/0077271 A1
(43) Pub. Date: Apr. 24, 2003

(54) Use of coagulation factor VII-activating protease for the prophylaxis and therapy of vasoproliferative disorders

(76) Inventors: Christian Kannemeier, Giessen (DE); Juergen Roemisch, Voesendorf (AT); Klaus T. Preissner, Giessen (DE); Sandip Kanse, Linden-Leihgestern (DE)

Correspondence Address: Finnegan, Henderson, Farabow,, Garrett & Dunner, L.L.P., 1300 I Street, N.W., Washington, DC, 20005-3315, US

(21) Appl. No.: 10/254,662

(22) Filed: Sep. 26, 2002

(30) Foreign Application Priority Data
Sep. 28, 2001 (DE) 101 48 037.7

(51) Int. Cl. A61K 38/48; C12N 9/64

(52) U.S. Cl. 424/94.64; 435/226

(57) ABSTRACT

The use of coagulation factor VII-activating protease (FSAP) for the prophylaxis and/or therapy of vasoproliferative disorders or oncoses is described. Reference is made in particular to the use of the protease for retinopathy, neuropathy, rheumatoid arthritis, psoriasis, endometriosis, bronchitis (especially chronic) or chronic inflammations in the gastrointestinal tract.

The invention relates to the use of the coagulation factor VII-activating protease for vasoproliferative disorders.

The German patent application 199 03 693.4 discloses a protease which is isolated from blood plasma and which is able to activate coagulation factor VII. Also previously described therein are a process for obtaining it, detecting it and inactivating it, and pharmaceutical preparations containing this protease. In accordance with its properties, this protease is referred to as factor seven activating protease (=FSAP). The German patent application 199 03 693.4 additionally reports that FSAP has a plasminogen activator activating and/or potentiating action which can be measured through the activation of single-chain urokinase (scu PA, single chain urokinase plasminogen activator) or single-chain tPA (sctPA, single chain tissue plasminogen activator).

The German patent application 199 03 693.4 has also described processes and test systems for the qualitative and quantitative detection of FSAP which are based on measurement of the action reducing the blood clotting times and the actions activating plasminogen activators of FSAP. Test systems of these types also allow FSAP to be measured in complex protein solutions such as plasma, as described in particular in the German patent application 199 26 531.3.

It has additionally been disclosed that FSAP influences hemostasis and the cellular processes connected therewith. Through involvement in blood clotting and/or fibrinolysis it also acts on the wound-healing response. Other previously disclosed characteristics are the strong binding to hyaluronic acid and glycosaminoglycans and the rapid autoactivation of the proenzyme in the presence of, for example, heparin or dextran sulfate. The property of binding to negatively charged surfaces, and the high affinity for heparin makes it probable that the protease interacts with cell surfaces and extracellular matrix. Characteristics of the cellular binding and resulting functional effects have not, however, previously been disclosed.

DE 100 52 319.6, the contents of which are hereby expressly included in this application, describes an FSAP mutant whose potency in activating factor VII is unimpaired or negligibly impaired. Mutants of this type are likewise subsumed under the term FSAP for the purpose of this application.

It has now been found that the proliferation of vessel wall cells can be significantly reduced by incubation with FSAP. This effect was observed especially in the presence of the activated protease. Inhibition of FSAP by, for example, aprotinin led to a reduction in this effect. Correspondingly, the proenzyme has only a weak effect on the proliferation of vessel wall cells.

The invention therefore relates to the use of coagulation factor-activating protease (FSAP) for the prophylaxis and/or therapy of vasoproliferative disorders or oncoses. The protease FSAP employed for this purpose can be isolated from blood plasma or be produced transgenically or recombinantly.

Cellular proliferation is a physiological process associated with growth processes of normal and transformed cells, which take place in an uncontrolled manner in tumor development. Dysregulation of the proliferation or division of cells can be inhibited by the existing property of the activated form of FSAP and can serve as prophylaxis or therapy of, for example, vascular disorders or lymphoproliferative diseases.

Proliferating smooth muscle cells of the vessel walls contribute, for example, to the development of arteriosclerosis which is regarded as preparing the way for the acute onset of thrombotic and thromboembolic complications such as myocardial infarction or pulmonary embolism. As illustrated in the example below, FSAP significantly reduces the proliferation of human smooth muscle cells when they are stimulated with a growth factor such as platelet-derived growth factor (PDGF) which is also involved in the pathogenesis of vascular disorders.

An increased proliferation of vessel wall cells is also observed after organ transplantations, where the immunological rejection can usually be prevented or delayed by immunosuppressing substances. The narrowing of vessels due to the proliferation of vessel wall cells leads to undersupply of the organ, to thrombotic complications and eventually to loss of the organ. Accordingly, FSAP might also be employed for the prevention and rejection after transplantations.

In addition, FSAP can also be used for the prophylaxis or therapy of oncoses in two ways: on the one hand, the uninhibited proliferation of tumor cells plays a dominant role in the development of cancer and, on the other hand, the proliferation of endothelial cells, and the formation of new vessels associated therewith (angiogenesis), contributes to the growth of many solid tumors in particular. A reduction in the growth both of the tumor cells and of the vascularization of the tumor via the antiproliferative effect of FSAP may lead to a reduction in the tumor mass, and thus potentially bring about cessation of growth or tumor regression. In a similar way, FSAP can be employed for the therapy of (vaso)proliferative disorders and endothelial proliferation associated therewith in, for example, retinopathy, neuropathy, rheumatoid arthritis, psoriasis, endometriosis, bronchitis (especially chronic), or chronic inflammations of the gastrointestinal tract.

### DESCRIPTION OF THE FIGURES

FIG. 1 shows the results of a proliferation test as a function of FSAP, and controls.

FIG. 2 shows the results of a proliferation test as a function of various pro-FSAP concentrations.

The antiproliferative effect of FSAP is shown by the following example:

### EXAMPLE

Proliferation Assay

The proliferation rate of human smooth muscle cells was carried out by means of a DNA synthesis test (Roche Molecular Biochemicals, Mannheim, Germany). For this purpose, 10 000 human smooth vascular muscle cells were seeded in each well of a 96-well microtiter plate. The cells were cultivated in Dulbecco's modified eagle medium (DMEM) with 10% fetal calf serum (FCS), 2 mM glutamine, 100 units/ml penicillin/streptomycin, 1 mM sodium pyruvate, 4 g/l glucose at 37° C. for two days and then incubated in DMEM without ingredients at 37° C. for a further two days. The cells were then transferred into DMEM with 0.2% FCS, 10 ng/ml platelet-derived growth factor bb (PDGFbb) and incubated in each case in the presence of the following reagents at 37° C. for a further 24 h: 5% FCS (a), Trasylol®, 100 units/ml (b), 0.8 m NaCl, 5 mM Na acetate, pH 4.5 (FSAP buffer), diluted 1:20 (c), FSAP buffer diluted 1:20 and 100 units/ml Trasylol® (d), FSAP (activated) in the concentrations of 31.3 μg/ml (e), 15.6 μg/ml (f) and 9.1 μg/ml (g), in each case with and without 100 units/ml Trasylol® (h-j). As control, a sample was tested without PDGFbb (k), or the above-described medium alone (I). In addition, pro-FSAP was tested with 25 μg/ml (m) 12.5 μg/ml (n) and 6.3 μg/ml (o) under the above-described conditions.

After an incubation time of 16 h, the DNA labeling reagent 5-bromo-2′-deoxyuridine (BrdU) was added in the dilution stated by the manufacturer to the reaction mixtures and, after an incubation time of 8 h at 37° C., the soluble cell supernatant was extracted, and the fixation was carried out as stated by the manufacturer. A difference from the manufacturer's protocol was that the wells on the entire microtiter plate were blocked with 3% BSA after fixation overnight at 4° C. The BrdU intercalated with the DNA was then detected with the aid of a peroxidase-labeled antibody in accordance with the manufacturer's statements.

Western Blotting:

DMEM with 0.2% FCS, 20 ng/ml PDGFbb was incubated at 37° C. with 2.5 PEU/ml FSAP or without protease. 75 μl samples were taken from the mixture at the times of 1 min, 10 min, 30 min and 100 min and mixed with 25 μl of 4-fold concentrated, nonreducing SDS sample buffer and denatured at 100° C. for 45 min. The samples were then loaded onto a 10% polyacrylamide gel 1 mm thick and fractionated in an SDS-containing running buffer at 100 volt for 1 h 45 min. The fractionated proteins were then transferred to a PVDF membrane (Amersham Pharmacia) in a Western blotting chamber (Bio-Rad, Germany) at 100 volt within 2 h and was subsequently saturated with 5% milk powder in 150 mM NaCl, 50 mM Tris, 0.1% Tween 20 at room temperature for 1 h. After the membrane has been incubated with a rabbit antibody, against PDGFbb (dilution 1:3 000) at 4° C. for 16 h, it was then washed 3×TBST and incubated with the detection antibody (pig anti-rabbit, 1:3 000) at 22° C. for one hour. After three further washing steps with TBST for 5 min each, an ECL substrate (Amersham Pharmacia, Sweden) was employed to detect the antibody binding, so that protein bands became visible after exposure to Hyperfilm X-ray films (Amersham Pharmacia) for 2 min.

Results:

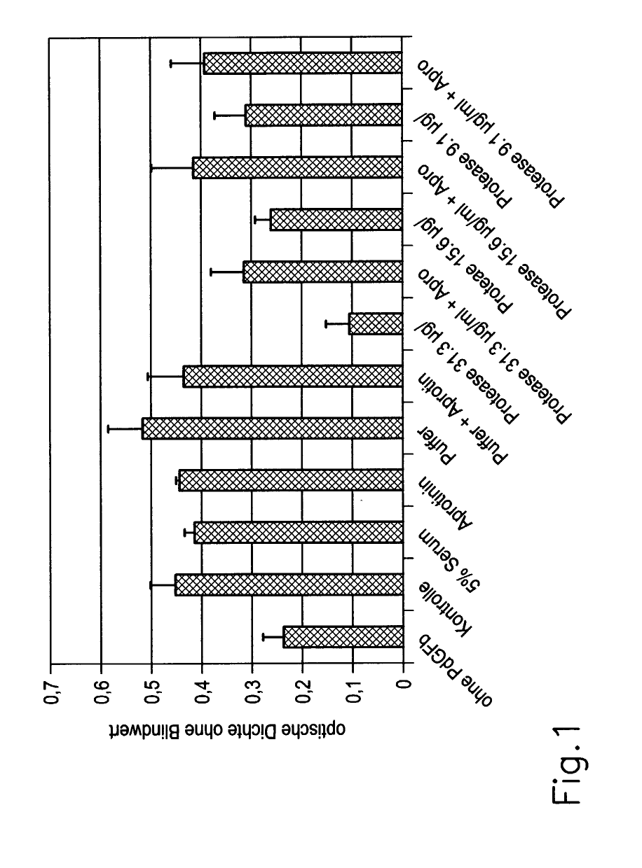

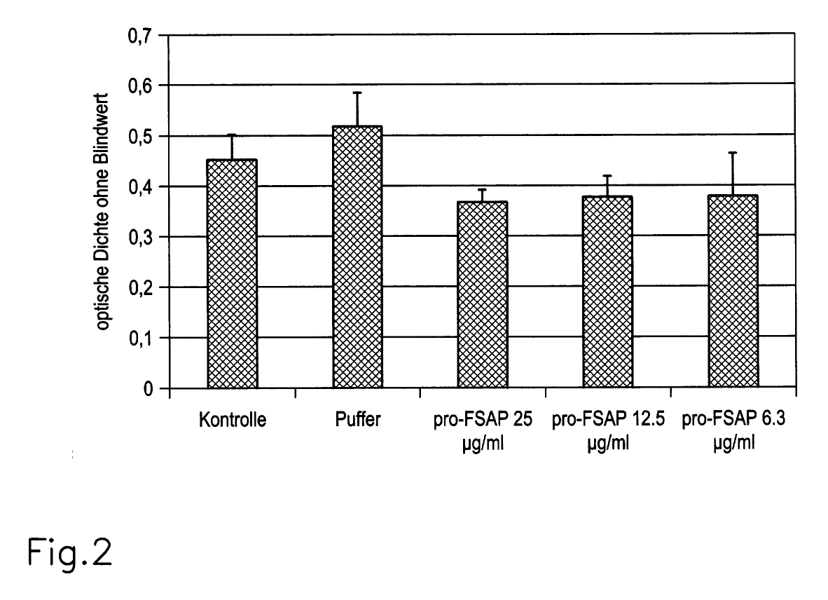

FIG. 1 shows by way of example the result of the proliferation test. FSAP inhibits in a concentration-dependent manner the proliferation of smooth muscle cells which is stimulated by PDGFbb, while buffer on its own shows no effect. This antiproliferative effect can be almost completely abolished by aprotinin. In contrast to the protease, pro-FSAP has only a small or no antiproliferative effect (FIG. 2). After coincubation of PDGFbb with FSAP, no cleavage of PDGFbb is to be seen in a Western blot (not shown), which precludes inactivation of the growth factor by FSAP. Accordingly, the FSAP effect appears to be attributable to a direct interaction of the protease with the cells, but a possible receptor is unknown. It was possible through this novel effect of the protease on smooth muscle cells to demonstrate a regulatory or inhibitory function of the protease, for example in various vasoproliferative processes.

The invention therefore also relates to a pharmaceutical preparation which comprises an amount of coagulation factor VII-activating protease which is sufficient for the prophylaxis and/or therapy of vasoproliferative disorders or oncoses.

DE 101 31 404.3, the contents of which are hereby expressly included in this application, describes stabilized liquid preparations which can-be used as pharmaceutical preparation.

1. The use of coagulation factor VII-activating protease (FSAP) for the prophylaxis and/or therapy of vasoproliferative disorders or oncoses.

2. The use of coagulation factor VII-activating protease (FSAP) as claimed in claim 1 for the prophylaxis and therapy of retinopathy, neuropathy, rheumatoid arthritis, psoriasis, endometriosis, bronchitis (especially chronic) or chronic inflammations of the gastrointestinal tract.

3. The use as claimed in claims 1 and 2, wherein a coagulation factor VII-activating protease which has been isolated from blood plasma or produced transgenically or recombinantly is employed.

4. A pharmaceutical preparation which comprises an amount of coagulation factor VII-activating protease which is sufficient for the prophylaxis and/or therapy of the disorders mentioned in claims 1 and 2.
